(19) United States
(12) Patent Application Publication
(10) Pub. No.: US 2026/0076795 A1
(43) Pub. Date: Mar. 19, 2026

(54) BIOLOGICALLY-ENGINEERED PEDIATRIC VALVED CONDUIT AND METHODS OF MAKING AND USING

(71) Applicant: Regents of the University of Minnesota, Minneapolis, MN (US)

(72) Inventors: Robert Tranquillo, Minneapolis, MN (US); Zeeshan Syedain, Minneapolis, MN (US)

(21) Appl. No.: 19/109,169

(22) PCT Filed: Sep. 15, 2023
(86) PCT No.: PCT/US2023/032921

§ 371 Date: Mar. 6, 2025

Related U.S. Application Data
(60) Provisional application No. 63/375,844, filed on Sep. 15, 2022.

(51) Int. Cl. A61F 2/24

(52) U.S. Cl. CPC A61F 2/2415

(57) ABSTRACT

A pediatric valved conduit, comprising: a biologically-engineered tubular conduit comprising a conduit inlet end, a conduit outlet end, and a conduit lumen therebetween; a biologically-engineered valve comprising a valve inlet end and a valve outlet end, wherein the biologically-engineered valve is positioned in the conduit lumen, wherein the valve inlet end of the biologically-engineered valve is fixedly attached in the lumen of the tubular conduit near the conduit inlet end and wherein the valve outlet end of the biologically-engineered valve is fixedly attached in the lumen of the tubular conduit near the conduit outlet end.

### CROSS REFERENCE TO RELATED APPLICATIONS

This application claims the benefit of priority under 35 U.S.C. § 119(e) to U.S. Application No. 63/375,844 filed on Sep. 15, 2022.

### FEDERALLY SPONSORED RESEARCH OR DEVELOPMENT

This invention was made with government support under HL107572 awarded by the National Institutes of Health. The government has certain rights in the invention.

### TECHNICAL FIELD

This disclosure generally relates to biologically-engineered pediatric valved conduit and methods of making and using.

### BACKGROUND

A heart valve that can grow and maintain function for pediatric patients has not yet been demonstrated. The only accepted options for these children are valves made from chemically-fixed tissues that often become dysfunctional due to calcification and often need to be replaced at least once since it has no growth capacity due to the chemical fixation.

These children usually endure several open heart surgeries until adulthood, when a mechanical valve typically would be implanted. If a valve could be implanted that is able to develop a functional endothelium and grow with the recipient, it would alleviate immense suffering for these children and their families, and reduce extremely high health care costs. Therefore, there is a dire need for a heart valve that can grow with a child.

### SUMMARY

In one aspect, a pediatric valved conduit is provided. Such a pediatric valved conduit typically includes a biologically-engineered tubular conduit comprising a conduit inlet end, a conduit outlet end, and a conduit lumen therebetween; a biologically-engineered valve comprising a valve inlet end and a valve outlet end, wherein the biologically-engineered valve is positioned in the conduit lumen, wherein the valve inlet end of the biologically-engineered valve is fixedly attached in the lumen of the tubular conduit near the conduit inlet end and wherein the valve outlet end of the biologically-engineered valve is fixedly attached in the lumen of the tubular conduit near the conduit outlet end.

In some embodiments, the conduit inlet end and/or the conduit outlet end of the biologically-engineered tubular conduit extend(s) at least about 3 mm (e.g., at least about 5 mm, at least about 10 mm, at least about 15 mm, at least about 20 mm, etc.) beyond the valve inlet end and/or the valve outlet end of the biologically-engineered valve, respectively.

In some embodiments, the biologically-engineered tubular conduit is about 8 mm to about 150 mm in length. In some embodiments, the pediatric valved conduit is about 10 mm to about 100 mm in length. In some embodiments, the diameter of the pediatric valved conduit is about 6 mm to about 24 mm.

In some embodiments, one or both of the ends of the biologically engineered valve is/are fixedly attached in the lumen of the tubular conduit via sutures or stitches, staples, adhesives (e.g. cyanoacrylate), double-sided adhesive tape, mechanical clips, crosslinking, chemical bonding, or thermal fusion (e.g., welding). In some embodiments, one or both of the ends of the biologically engineered valve is/are fixedly attached circumferentially in the lumen of the tubular conduit

In some embodiments, the biologically engineered tubular conduit and/or the biologically engineered valve comprise(s) predominantly type I collagen and/or type III collagen. In some embodiments, the type I collagen and/or type III collagen in the biologically engineered tubular conduit and/or the biologically engineered valve originates from thrombin, fibrinogen, and matrix-producing cells. In some embodiments, the matrix-producing cells are dermal fibroblasts.

In some embodiments, the biologically engineered tubular conduit is a decellularized biologically engineered tubular conduit. In some embodiments, the biologically engineered valve is a decellularized biologically engineered valve.

In another aspect, methods of repairing congenital heart defects are provided. Such methods typically include implanting the pediatric valved conduit as described herein into a pediatric subject suffering from a heart defect.

In some embodiments, the congenital heart defect is Tetralogy of Fallot (TOF), pulmonary valve stenosis, bicuspid aortic valve, aortic stenosis, transposition of greater arteries, leaflet prolapse, any form of Right Ventricle Outflow Tract (RVOT) obstruction that requires reconstruction (e.g., pulmonary atresia) or any type of defective cardiac valve in need of repair.

In some embodiments, the implanted pediatric valved conduit increases in size as the pediatric subject increases in size. In some embodiments, the pediatric subject is a neonate.

In some embodiments, the biologically engineered valve is selected from the group consisting of a mitral valve, an aortic valve, tricuspid valves, pulmonary heart valves, and vein valves. In some embodiments, the valve is a bi-leaflet valve. In some embodiments, the valve is a tri-leaflet valve.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIGS. 1A-1E are schematics showing a method of making a biologically engineered valve as described herein.

FIG. 2A-2D shows a biologically engineered valve as described herein. (2A) Schematic showing construction of a tri-leaflet valve from three biologically-engineered tubes and degradable suture. (2B) Exterior view of the sewn valve. Comparison of the new tri-tube valve (2C) and existing tube-in-tube valve (2D), with tubes color-coded for clarity and commissures circled to facilitate comparison of how diastolic force on the commissures is born entirely by the connecting sutures for the tube-in-tube valve (2C) and predominantly by the tube material for the tri-tube valve (2D).

FIG. 3 shows a Gen 3 valve (19 mm) implanted into the pulmonary annulus of a lamb (PACV22).

FIG. 4A-4B shows a CT-angiography reconstruction of LPA-1. 3-mo (FIGS. 4A) and 6-mo (FIG. 4B) images with overlay of graft diameter measurements. Note images are not identically scaled for display.

FIG. 5A-5C shows a schematic of Gen3 valved conduit construction process. FIG. 5A shows the tube trimming. Each tube is manually trimmed to create leaflet as shown. The front surface as shown becomes one leaflet, while the back surface becomes one-third of the conduit wall, after tri-tube stitching. FIG. 5B shows the tri-tube stitching. Three trimmed tubes are stitched together into a closed ring along the long axis where they contact using Maxon 7-0 sutures (Medtronic) with each suture throw ˜2 mm apart. A stitch line is then formed running along the circumference of all three tubes (closing the bottom of each tube and forming a leaflet of 11 mm height). The valve is thus fully stitched with a total of 4 running sutures and 4 sets of knots. FIG. 5C shows the outer sleeve. The stitched tri-tube valve (˜20 mm outer diameter) is inserted inside a tube of slightly larger diameter (22 mm) for close contact between the two. Its length is 9 cm long. Using 6-0 Maxon suture, the sleeve is stitched around the inlet and outlet circumference of the tri-tube valve, positioned in the center of the sleeve (thus extending ˜3 cm past the valve at both ends), with running sutures using a total of 2 running sutures and 2 sets of knots.

FIG. 6 shows a 12-week echocardiogram following implantation of the valved conduit (diastole (6A); systole (6B); inlet (6C)) and a photograph of the valved conduit showing direct myocardial anastomosis (6D).

Like reference symbols in the various drawings indicate like elements.

### DETAILED DESCRIPTION

We have developed a biologically-engineered tube of cell-produced collagenous matrix that has unprecedented growth potential for repair of congenital heart defects. The tube is grown from donor dermal fibroblasts entrapped in a sacrificial fibrin hydrogel tube that is then decellularized using sequential detergent treatments and storable in cold sterile saline solution. The resulting cell-produced matrix tube is thus non-immunogenic and “off-the-shelf.” It possesses physiological strength, compliance, and alignment (circumferential). We have shown it becomes a living artery-like vessel, populated by the recipient's cells, and grows in size like the adjacent native artery when implanted into the pulmonary artery (PA) of a lamb as it grows into adult sheep size over a year. Moreover, there was no evidence of macro-calcification or overt systemic immune response. We have also demonstrated that we can grow tubes from human fibroblasts that function for at least 6 months in a human arterio-venous graft model (baboon) without overt systemic immune response.

Using these tubes and the concept of a tubular heart valve, where the tube collapses inward with back-pressure between 3 equi-spaced constraints placed around the periphery to create one-way valve action, we have reported unprecedented results for heart valve tissue engineering implanting valves fabricated from these tubes mounted on 3-pronged crown frames into the sheep aortic position for 6 months. Most recently, a tri-tube valve design with superior commissures has been developed, with valves functioning in a growing lamb for one year, unlike clinically-used valves implanted in the same lamb model whose function (e.g., effective orifice area, systolic pressure drop) deteriorated in much shorter time due to gross calcification. An increase in both leaflet size and root diameter occurred at one year, indicating functional valve growth. These promising preclinical results may lead to transformative therapy for congenital heart disease when a defective vessel or valve of the heart must be replaced, since the need for multiple open-heart surgeries could be eliminated using unvalved and valved conduits made from this unique biologically-engineered tube.

Described herein is a subsequent clinical trial of our valved conduit that can be used to correct more common cases, but mechanically a much more demanding application, such as Tetralogy of Fallot (TOF)-a common congenital heart defect that includes obstructed outflow of blood from the right ventricle to the lungs because of a malformed pulmonary artery and valve, a.k.a. right ventricular outflow tract (RVOT). According to the National Organization for Rare Disorders, approx. 0.033% of newborns are diagnosed with TOF, which translates to approx. 1,300 cases per year in the U.S. Thus, our goal is to produce a valved conduit using our tri-tube design fabricated from our biologically-engineered tubes, already tested in growing lambs, in adolescent TOF patients who have a prognosis for at least two more valve replacement reoperations. Ultimately, we seek to produce a 10-12 mm tri-tube valved conduit suitable for surgical repair of TOF in newborns/infants.

As described in more detail in U.S. Pat. No. 11,589,982, which is incorporated by reference herein in its entirety, biologically engineered tissues such as, without limitation, tubes, can be generated in vitro using extracellular matrix (ECM)-producing cells (e.g., fibroblasts, e.g., dermal fibroblasts) within a gel containing fibrin or the like. Over a period of time (e.g., 7 weeks, 8 weeks, or 9 weeks) and under appropriate culture and mechanical manipulation conditions, the ECM-producing cells convert the gel material (e.g., fibrin gel) into a circumferentially-aligned dense collagenous extracellular matrix (ECM), which then can be decellularized using detergents, dehydrated (e.g., freeze drying), or fixed / crosslinked (e.g., glutaraldehyde fixation) to create a biologically engineered graft (e.g., an allograft) with or without cells that can be stored for several months without loss of mechanical properties. The resulting biologically engineered tissue doesn't require any type of crosslinking, and, when decellularized, doesn't cause an immune response. See, also, Syedain et al., 2011, Biomaterials, 32(3):714-22; or Dahl et al., 2011, Sci. Transl. Med., 3(68):68ra9.

The biologically engineered tissues produced in this manner exhibit a non-linear stress-strain curve typical of native tissues and possess physiological compliance and a burst pressure that meets or exceeds that of native vessels. The resulting tensile mechanical properties compared well to ovine pulmonary valve leaflets in terms of modulus and ultimate tensile strength (UTS) measured in strain-to-failure testing. The same is also true for collagen content. In addition, it has been shown in sheep and in baboons that these grafts become populated by appropriate host cells, including endothelium formation, without a sustained inflammatory response, overt immune response, or calcification of arterial grafts or tubular heart valves.

In addition, the biologically engineered tissues described herein have been shown to grow with young lambs into adulthood. Since no structural element (e.g., metal or plastic frame) is required, the biologically engineered tissues described herein are not structurally constrained and can allow for growth, particularly when, for example, degradable sutures or the like are used. The use of a fibrin gel as the starting material allows it to be molded or shaped as desired, and, with respect to tubes, allows the molding or shaping of virtually any length and diameter of a tube. Tubular members of various diameters can be used to produce a given target diameter for the valve, which will lead to leaflets having different areas. These features are particularly significant as they pertain to pediatric applications and valve performance under specified pressure-flow conditions.

In addition, a tubular conduit can be produced in the same manner as the tubes used to generate the valve. A tubular conduit can be used to provide connections on either or both ends of a valve to facilitate surgical connection and attachment of the valve (e.g., from the heart to the target vessel, e.g., from the pulmonary annulus to the main pulmonary artery in RVOT reconstruction). A tubular conduit can having virtually any diameter, but should be a suitable diameter such that the valve fits within the lumen of the tubular conduit.

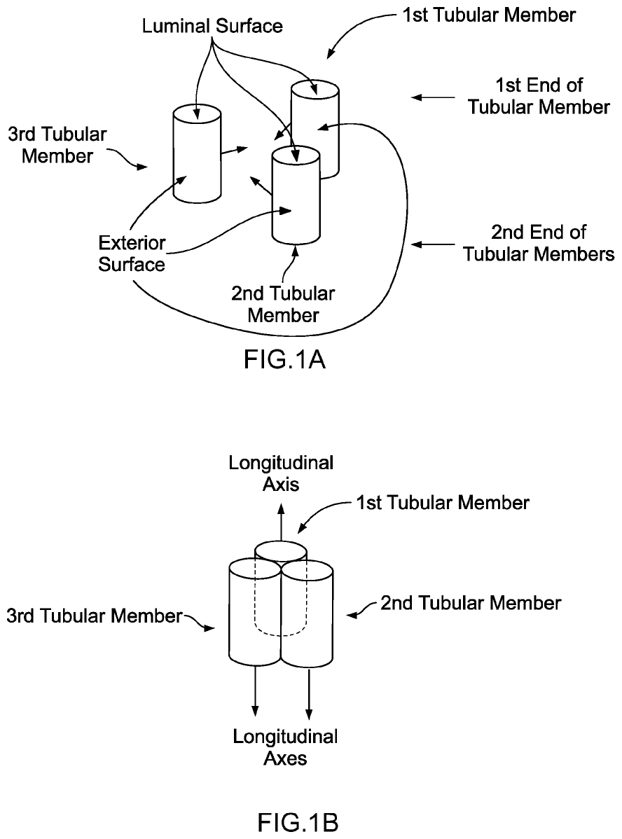

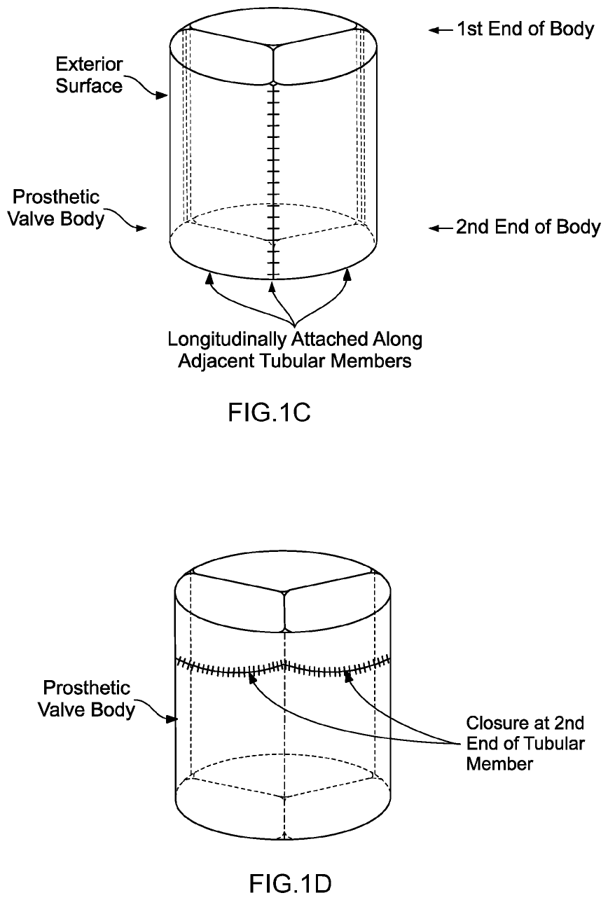

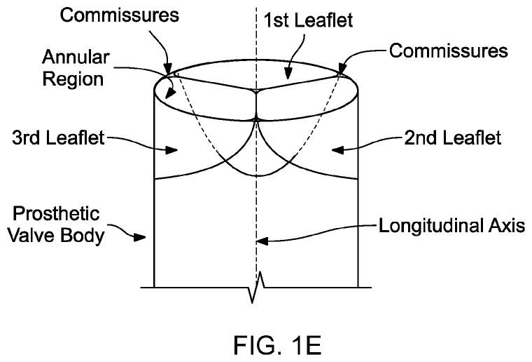

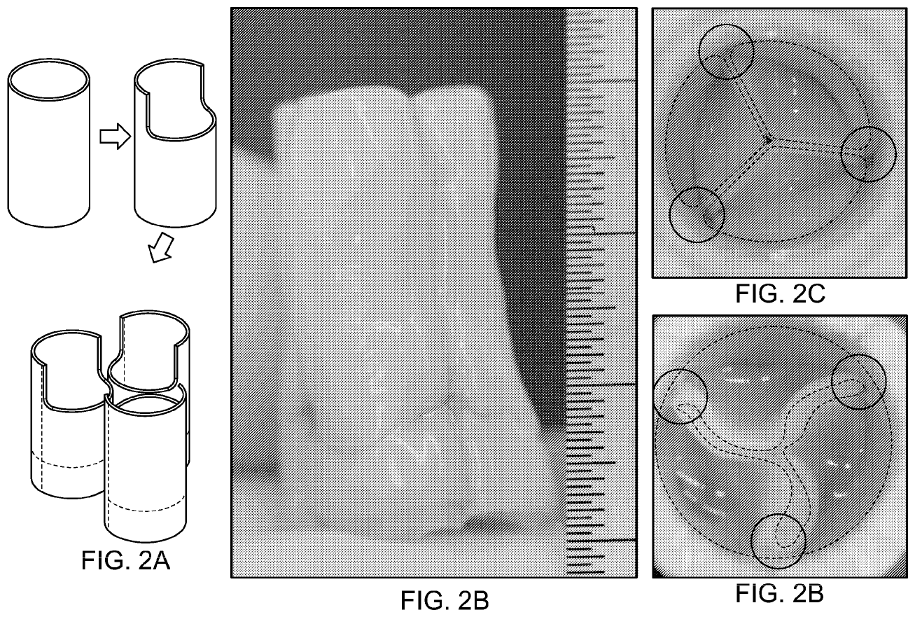

Referring to FIG. 1, a biologically engineered valve that starts with three tubular members is shown, which ultimately results in a tri-leaflet biologically engineered valve. However, it should be appreciated that the techniques described herein (and represented schematically in FIGS. 1A-1E) also can be applied to create a bi-leaflet biologically engineered valve (i.e., starting with two tubular members) or a quad-leaflet biologically engineered valve (i.e., starting with four tubular members). Thus, the particular configuration of the tri-leaflet biologically engineered valve shown in FIGS. 1A-1E, as well as in FIG. 2, should be considered exemplary. Specifically with respect to FIG. 1A, three tubular members are shown and correspond to the starting material. As shown in FIG. 1A, each tubular member includes a first end and a second end, and has an exterior surface and a luminal surface (i.e., the inside surface of each tubular member), which define a longitudinal axis. As shown in FIG. 1A, the tubular members are positioned parallel to one another (i.e., aligned along their longitudinal axes).

With respect to FIG. 1B, the aligned tubular members in a biologically engineered valve are brought into contact with one another, and FIG. 1C shows that each of the tubular members are attached to the adjacent tubular member(s) along a longitudinal seam. The longitudinal seam is located between the adjoining exterior surfaces of each tubular member. As shown in FIG. 1C, portions of the exterior surface of the adjoined tubular members form a circumferential wall of a prosthetic valve body. Similar to each tubular member, the body of the valve has a first end and a second end through which a longitudinal axis passes, as well as an outer surface and an annulus.

FIG. 1D shows that each tubular member in the biologically engineered valve is then closed at its second end. Closure at the second end of each tubular member creates a leaflet (or a cusp), with the top surface of each leaflet (i.e., the surface at the first end of the valve body) formed from the luminal (or inside) surface of each tubular member (FIG. 1E). The bottom surface of each leaflet (i.e., the surface that projects toward the second end of the valve body) is formed from a portion of the exterior surface of each adjoined tubular member that does not form part of the circumferential wall (FIG. 1E).

Each leaflet has both a commissure region and an annular region. As shown in FIG. 1E, the portions of the exterior surface of the adjoined tubular members that do not form part of the circumferential wall of the body, at the first end, define the commissures of the valve, while the luminal surface of each tubular member that is opposite the exterior surface of each tubular member that forms the circumferential wall of the body, at the first end, defines the annular region of each leaflet. Due to the tubular structure by which each leaflet is generated, the commissure region and the annular region of each leaflet is contiguous (or integral with one another), and each leaflet, including the commissure and the annular region are formed by a single tubular member.

In some embodiments, the individual tubular members within a biologically engineered valve can be made from different materials. For example, in a bi-leaflet valve, one of the tubular members used in the starting materials can be a biologically engineered tubular member while the other tubular member used in the starting materials can be a native tissue tubular member. In another example, in a tri-leaflet valve, one of the starting tubular members can be a native tissue tubular member and the other two starting tubular members can be biologically-engineered tubular members. Virtually any combination of tubular members is envisioned and would be considered biologically engineered.

FIGS. 2A-2B shows another embodiment of a biologically engineered valve in which three tubes are sutured together in a closed ring and then the bottom of each tube is then sutured closed to form its own leaflet (only two of the axial suture lines connecting the three tubes together can be clearly seen, along with the complete closure of one of the tubes, creating one of the “leaflets”)

The design of a biologically engineered valve eliminates the most common site of failure of a valve made by suturing one tube inside another, where the load on the leaflets is transferred directly to the sutures used to attach the inner tube to the outer tube at the commissures. In the design described herein, the commissure is created by adjacent tubular members, so the load on the leaflets is carried by the tubular member itself and not the sutures. Thus, “suture pull-out,” which is another common problem with many of the currently used prosthetic valves, should not occur with the biologically engineered valves described herein.

One of the crucial differences in the commissures with this valve design is evident in comparing the connection of the three tubes in the current valve design (see FIG. 1 and FIGS. 2A, 2B and 2D) with the connection of the two tubes in the original tube-in-tube design (see FIG. 2C), where each color represents a tube that was sewn together to form the valves and the circles indicate the commissure locations. It would be understood that downward force on the leaflets is going to be predominately carried by the bulk matrix in the current valve design (FIG. 2D) as opposed to the suture in the original tube-in-tube design (FIG. 2C).

As described herein, once the biologically engineered tubular conduit and the biologically engineered valve are produced, the biologically engineered valve can be inserted into the lumen of the biologically engineered tubular conduit and fixedly attached in order to fabricate the biologically engineered valved conduit. It would be understood that the inlet end of the biologically-engineered valve (referred to as the “valve inlet end”) is fixedly attached in the lumen of the tubular conduit near the inlet end of the tubular conduit (referred to as the “conduit inlet end”). Similarly, it would be understood that the outlet end of the biologically-engineered valve (referred to as the “valve outlet end”) is fixedly attached in the lumen of the tubular conduit near the outlet end of the tubular conduit (referred to as the “conduit outlet end”). In some embodiments, the biologically engineered valve is fixedly attached around the circumference of the lumen of the biologically engineered tubular conduit.

Based on the disclosure herein, it would be appreciated that the length of the biologically engineered tubular conduit usually is longer than the length of the biologically engineered valve. For example, the conduit inlet end and/or the conduit outlet end of the biologically-engineered tubular conduit can each extend at least about 3 mm (e.g., at least about 5 mm, at least about 10 mm, at least about 15 mm, at least about 20 mm, etc.) beyond the valve inlet end and/or the valve outlet end of the biologically-engineered valve, respectively. In some embodiments, the biologically-engineered tubular conduit is about 8 mm to about 150 mm in length (e.g., about 12 mm to about 100 mm in length; about 15 mm to about 75 mm in length; about 20 mm to about 50 mm in length; or about 25 mm to about 30 mm in length). In some embodiments, the diameter of the pediatric valved conduit is about 6 mm to about 24 mm (e.g., about 8 mm to about 20 mm in diameter; about 10 mm to about 15 mm in diameter; about 12 to about 16 mm in diameter; about 15 to about 20 mm in diameter).

Connecting or joining tissues and the various connectors for doing so are known in the art and include, without limitation, sutures or stitches, staples, adhesives (e.g. cyanoacrylate), double-sided adhesive tape, mechanical clips, crosslinking, chemical bonding, or thermal fusion (e.g., welding). Such connectors can be used to attach tubular members to adjacent tubular members along a longitudinal seam in the biologically engineered valve, to close each tubular member at the second end of the biologically engineered valve, and/or to fixedly attach a biologically engineered valve inside the biologically engineered tubular conduit to produce a valved conduit as described herein.

Regenerative processes result in recellularization and new matrix deposition within our biologically-engineered tri-tube valve conferring its growth and self-healing potential.

These attributes make the relevant pre-implantation testing of this valve different from prosthetic valves materials. For example, passive durability (e.g., 200M cycles, approx. 5.5 years in vivo) in accelerated wear testing is relevant for inert materials, but the tri-tube valve begins regenerating within months, evident from new collagen and elastin deposition, and is expected to fully regenerate in much less than 5.5 years. Simply by way of example, the following methods and assays can be used to evaluate a valved conduit as described herein.

### Biological Safety

The biologically-engineered tube comprised of allogeneic extracellular matrix that is used to construct the valve via stitching has been evaluated in a non-human primate (baboon) model and in humans as vascular graft for up to 6 months (Syedain et al., 2017, Sci. Transl. Med., 9:414; Ebner et al., 2023, J. Vasc. Access, doi: 10.1177/11297298221147709), including immunological evaluation (using a Panel Reactive Antibodies (PRA)), histopathology, regeneration and gross necropsy. Its safety in humans would be assessed in the aforementioned clinical trial of surgical repair discontinuous pulmonary arteries and hemitruncus in neonates and infants that would precede this clinical trial of the valved conduit constructed from the tube.

### Material and Mechanical Property Testing

The 16 mm diameter biologically-engineered tube is released from GMP manufacturing with lot release properties including suture retention and burst strength tests. Representative 19 mm valves made from the 16 mm tubes tested have been evaluated for hydrodynamic and durability testing prior to implantation in a lamb model (Syedain, 2021, Sci. Transl. Med., 13:585).

### Hydrodynamic Performance Assessment

Pediatric heart valve conditions have been used to evaluate hydrodynamic performance of the valve conduit. The performance criteria for pediatric valves has the smallest diameter as 17 mm, which is intermediate to the smallest (˜11 mm) and largest (19 mm) valve sizes anticipated for clinical use. The valve will be evaluated at the stated specifications for 19 mm. Although no specifications exist for smaller diameter valves, they will be subject to similar testing.

Pediatric heart valve right side conditions matched to relevant valve diameter are used (e.g. “adolescent” conditions for the initial 19 mm valved conduit and “newborn/infant” for the subsequent ˜11 mm valved conduit), with a matched diameter bovine jugular vein valve (expired Contegra™ valve) as the reference valve.

There are no performance criteria for pediatric valves. For the 19 mm valves, minimum device performance requirements for Adult Aortic Valves are used: EOA<0.85 cm², TRF<10% at beat rate=70 cycles/min, simulated CO=5 L/min, and systolic duration=35 %, at normotensive pressure. Also, mean and systolic pressure drops must be equal to or less than measured for the reference valve.

### Implant Durability Assessment

The biologically-engineered matrix tubes comprising the valve are intended to regenerate within the patient's body, i.e. the material is not inert as for traditional bioprosthetic heart valves. As such, an integrated approach to durability assessment is proposed. The stitched valve is accelerated wear tested to failure with precautions taken to minimize microbial contamination and potential matrix degradation (since the collagenous matrix is not cross-linked). This information combined with valve function in the extended-term studies in the growing lamb model further supports the durability of the biologically-engineered valve.

Accelerated wear testing (AWT) will be performed with a minimum pulmonary peak differential pressure of 10 mm Hg (pediatric right side valve, all age groups). Assessment of durability also is performed in the preclinical evaluation. It is expected that durability is conferred by recellularization and matrix remodeling post-implantation, as indicated by preclinical testing of up to 1-year duration (without material or suture line failure). Therefore, AWT is performed, but no acceptance criteria are applied.

### Fatigue Assessment

Unlike traditional bioprosthetic heart valves that are inert, in vivo fatigue evaluation is most relevant for the biologically-engineered valve. The material shows fatigues resistance in vivo for at least 52 weeks (˜37M cycles) in both the root and leaflets, which exhibit extensive and partial recellularization, respectively, at that time point. Given the valve design, which involves only stitching of the tubes together, the other most relevant fatigue assessment is AWT of the valve itself. We address fatigue resistance by Finite Element Analysis (FEA) analysis of the valve based on the mechanical properties of the initial matrix.

FEA of the valve is performed to estimate peak stress magnitude and location during a representative pressure waveform of a cardiac cycle. Peak stress must be <15% of UTS of the implanted matrix (safety factor of 6).

### Thrombogenic and Haemolytic Potential Assessment

Fluid-Structure Interaction (FSI) simulations using a Casson blood rheology approximation is performed to assess potential regions of blood stagnation (near-zero shear rate) behind the leaflets and adverse shear stress on the root and leaflets. Acceptance criteria include no substantial difference of blood residence time distribution for flow volume behind leaflets (below the flow separation point) or OSI/TAWSS patterns on leaflet outflow surface compared to native pulmonary valve during a cardiac cycle.

### Preclinical in Vivo Evaluation

Valve function (longitudinal TTE) and remodeling / recellularization (explant tensile mechanical testing, histology, immunocytochemistry, and biochemical analysis) were evaluated at 52 wk (n=3) in the Gen 3 tri-tube valved conduit results in the growing lamb, plus the existing Gen 1 results (n=4, including 52 wk) and Gen 2 results (n=3 at 52 wk).

Acceptance criteria includes maintained function (pulmonary insufficiency less than moderate, systolic pressure gradient <10 mm Hg) and increase in conduit/valve diameter within one standard deviation of normal age-matched lambs, recellularization of phenotype-appropriate cells, and no macroscopic calcification or thrombus.

### Sterilization

The biologically-engineered tube is developed in an aseptic culture process (with no antibiotics), with validated sterilization of released tube batches. The valved conduit will be assembled/stitched using sterile instruments in a laminar hood located in a cleanroom. The final stitched valve will be kept in sterile buffer solution with solution evaluation for sterility by USP immersion test.

### Flow Testing

Steady and pulsatile flow testing is performed for the largest (19 mm) and smallest (10-12 mm) valve sizes. Pediatric heart valve right side conditions matched to relevant valve diameter will be used (e.g. “adolescent” conditions for the initial 19 mm valved conduit and “newborn/infant” for the subsequent ˜11 mm valved conduit), with a matched diameter bovine jugular vein valve (expired Contegra™ valve) as the reference valve. Acceptance criteria requires any closing volume and forward flow pressure drop (at every flow rate) to be no greater than the reference valve.

A pediatric valved conduit as described herein can be implanted into a pediatric subject (e.g., infants) to repair a congenital heart defect. As described herein, the valved conduit can grow with a subject, so is very well suited for pediatric subjects. Representative congenital heart defects include, without limitation, Tetralogy of Fallot (TOF), pulmonary valve stenosis, bicuspid aortic valve, aortic stenosis, transposition of greater arteries, leaflet prolapse, any form of Right Ventricle Outflow Tract (RVOT) obstruction that requires reconstruction (e.g., pulmonary atresia) or any type of defective cardiac valve in need of repair. As discussed herein, a valved conduit can be a bi-leaflet valve, a tri-leaflet valve, or a quad-leaflet valve; accordingly, it would be appreciated that a valved conduit as described herein can be used to replace or repair any of the cardiac valves (e.g., mitral, aortic, tricuspid, pulmonary).

In accordance with the present invention, there may be employed molecular biology, microbiology, biochemical, and recombinant DNA techniques within the skill of the art.

Such techniques are explained fully in the literature. The invention will be further described in the following examples, which do not limit the scope of the methods and compositions of matter described in the claims.

### EXAMPLES

### Example 1—Clinical Plan

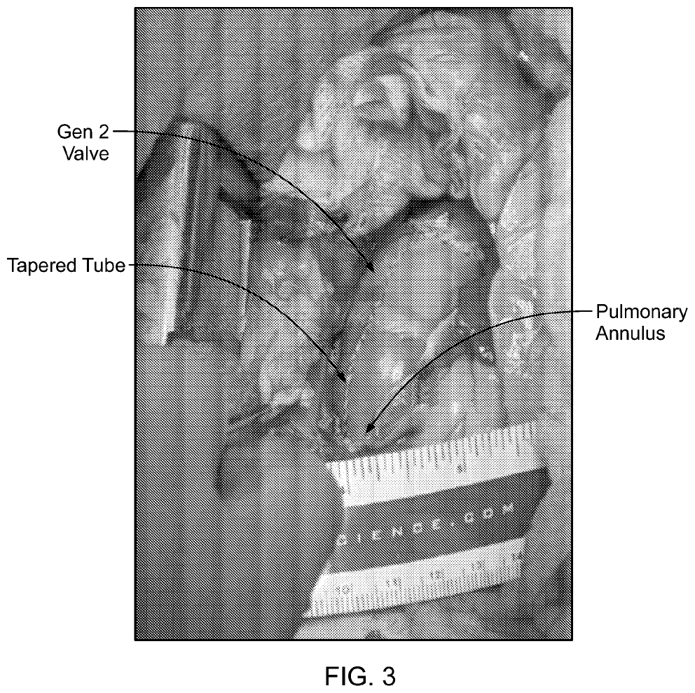

We implanted n=3 Gen 3 tri-tube 19 mm valves directly into the pulmonary valve annulus/myocardium using the same growing lamb model for a duration of 52 wk. Two of these implantations occurred with no valvular regurgitation at 1 month p.o. and the animals remain asymptomatic to date. The Gen 3 valve is the Gen 2 valve with a longer sleeve, providing an inflow conduit proximal to the valve to allow for anastomosis to the pulmonary annulus per clinical practice with the Contegra™ valve (FIG. 3). Longitudinal echocardiography and histological and mechanical testing assessment of the explanted valves was performed, with a focus on regeneration of the added tapered tube, particularly the anastomosis with the pulmonary valve annulus, which is the distinguishing feature of the Gen 3 implant. As described herein, a Gen 3 valved conduit is a Gen 2 valve sewn at both its ends within a sleeve (a tube of slightly larger diameter than the Gen 2 valve outer diameter) that extends out at least ˜3 cm from both ends of the valve, allowing the surgeon to cut each end of the valved conduit to the desired shape and length for the anastomoses per clinical practice using the Contegra™ valve.

16 mm tubes manufactured under GMP that meet FDA requirements are produced for product testing and for one year storage stability. The 19 mm valves are sewn using aseptic manufacturing in a laminar hood per current practice. Further environmental controls and additional QC oversight are performed to ensure compliance of valve manufacturing with FDA guidance on cGMP and standards regarding valve product sterility. Further valve hydrodynamic testing and (if necessary) accelerated wear testing are performed at an independent GLP lab.

6 mm tubes are manufactured under GMP for surgical repair of discontinuous pulmonary arteries and hemitruncus in neonates and infants. Tube diameters can be about 6-16 mm, which might be extended to 6-24 mm to include the sleeve used in the valved conduit.

Pending positive results from the use of the tubes as non-valved conduits for repair of discontinuous pulmonary arteries and hemitruncus, we intend to proceed with the use of the 19 mm Gen 3 valved conduit for surgical repair of TOF in adolescents and a 10-12 mm Gen 3 valved conduit for neonates.

### Example 2—Preclinical Studies

The primary mechanism of action of this product, a valved conduit constructed from tubes of extracellular matrix, is integrated and grows with the developing pulmonary trunk of patients with TOF. As a result, this product is expected to eliminate the need for repeated open heart surgeries to up-size the current non-growing valved conduits used.

We have completed several in vivo studies using an unvalved conduit (single tube of the biologically-engineered matrix), with additional animal studies ongoing. The nonclinical studies completed to date have demonstrated: recellularization of the matrix and maintained patency up to 6 months in the femoral artery of adult sheep; functionality of a tubular valve in the aortic valve location, with matrix remodeling and recellularization, in adult sheep for up to 6 months; integration and growth of an unvalved conduit into the pulmonary artery of juvenile lambs growing into adulthood (44 weeks); integration and growth of valved conduits constructed from the grafts implanted into the pulmonary artery of juvenile lambs growing into adulthood (62 weeks); and recellularization and patency of the tube made from human cells implanted in adult baboons as AV grafts without immunosuppression (6 months).

Nonclinical assessment of the RT1 product in animals is complicated by several factors. (1) All animal species used for medical device testing (including non-human primates (NHP)) have a faster growth rate than humans. (2) While the final product is acellular, the extracellular matrix is composed of human collagen (e.g., predominantly type I collagen and/or type III collagen), which can be immunogenic in animals; thus, the graft made for an animal study generally cannot be made from nHDF used for RT1 with the exception of old world NHP, which includes baboons and macaques. (3) No single graft placement in the chosen can replicate the range of anatomical placements of the graft that is expected to occur clinically. Therefore, the strategy for nonclinical assessment of this product required that some studies include modifications to the test article used and the surgical approach. The characteristics of the product that correspond to the proposed clinical use are as follows: size of tubular graft is 6 and 16 mm diameter with a length of 19±1 and 8±2 cm, respectively, and 0.2-0.6 mm thick; source of cells is allogeneic dermal fibroblasts (ovine cells for lamb studies, human cells for non-human primate studies); anatomical placement in the pulmonary trunk of neonates and infants with discontinuous branching pulmonary arteries or hemitruncus.

### Example 3—Simulating the Pulmonary Artery Branch Reconstruction in a Lamb

Tubes are implanted in a growing lamb model. Instead of interpositional implantation of a 16 mm tube into the main PA, interpositional implantation of a 6 mm tube into the left PA branch is performed in lambs of similar age (4 month old). To eliminate possible complicating xenogeneic reaction, the tubes used in this study are grown from ovine fibroblasts and then decellularized and characterized. A mixed sex/breed is used. Grafts are implanted while on cardiopulmonary bypass and harvested after 3 months (n=3) and 6 months (n=3) of implantation. A control group (n=3) implanted with a 6 mm Gortex tube is harvested after 6 months of implantation. Animals are examined at day 7, 3 months, and 6 months using transthoracic echocardiography (TTE) and CT-angiography to evaluate graft performance and size as well as heart function (the graft cannot be imaged with TTE for the duration, becoming obscured by the lungs during growth). Catheterization to obtain the pressure gradient across the graft and to perform angiography for the graft length and diameter, and patency assessment are conducted on the 6-month animals to demonstrate direct patient benefit. Grafts explanted from the 3-month and 6-month animals are examined histologically (H&E, trichrome, von Kossa) for assessment of longitudinal recellularization and integration of the grafts. Blood samples are collected monthly to perform a “ruminant combo,” which includes hematology and clinical chemistries. As in our prior studies in the sheep femoral artery model, enoxaparin is used mitigate blood clotting as a failure mode when the goal is to examine graft growth and since clotting is being assessed in the more relevant macaque model. Finally, the animals are euthanized and a full necropsy is performed (6 month animals only).

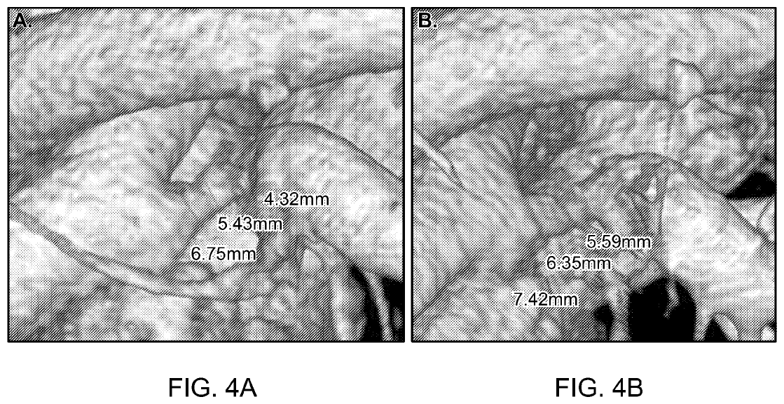

The main results to date are for two lambs, aged 5 wk, that have been implanted with a 6 mm graft. The lengths of the two 6 mm diameter grafts implanted into the LPA (following resection) are 10 mm and 7 mm, the maximum lengths possible as determined by the anatomy. Results for the first lamb are shown in FIG. 4. The cross-sectional lumen area along the graft based on CT-angiography at 3 mo and 6 mo increased 86%, consistent with graft diameter increases evident in both axial and sagittal planes (36% and 29%, respectively). The length also increased ˜2 mm (˜20% over the implant length of 10 mm). Interestingly, there was no increase evident in the RPA diameter over this time, while the main PA diameter increased 9%. The animal weight increased 26% during this period. The intended duration of these two animals is 18 months. One has survived to 12 months at this time, exhibiting further increase in graft dimensions.

A related study has commenced, with 7 lambs implanted. One animal that was implanted with a control (GoreTex) graft eventually died due to peritonitis and one animal implanted with a tube graft died in opero due to reasons deemed unrelated to the graft (i.e. due to surgical procedure). Three animals, all implanted with tube grafts, remained asymptomatic and were euthanized for a planned 3-month assessment of conduit recellularization. The other two animals, both with tube grafts, are asymptomatic to date.

Four more animals are scheduled for implantation surgery, which will be combined with the two ongoing animals to yield control (n=3) and tube (n=3) grafts survived to 6 months. Two additional animals may be implanted with tube grafts to yield n=5 for the test group.

### Example 4—Assessment of 4-Tube (Sleeved) Valved Conduit in 5 Juvenile Lambs for up to 52 Weeks

An additional nonclinical study in 3 juvenile lambs (PACV17-19), using a conduit fabricated from 4-tubes, was completed with a 52 week evaluation. This conduit is the same configuration as Gen1, with a 4^{th} tube placed as a ‘sleeve’ on the outside of the valve, referred to as “Gen2” valves. Two lambs were explanted at 52 week with mild/moderate regurgitation and one lamb progressed to moderate regurgitation between 44 and 52 weeks, when it was explanted. As for the Genl explanted valves, all leaflets maintained their initial height, but the leaflets of the Gen2 valves had a longer free edge length. This may reflect leaflet growth associated with their partial recellularization and the improvement in performance as the valve diameter increased from its implanted value of 19 mm to approximately 25 mm over 52 weeks. As for the Gen1 explanted valves, calcification was only occasional, sparse, and micropunctate. Two additional lambs were implanted with Gen2 valves (PACV20-21) with a target explantation of 104 weeks, but these valves progressed to severe regurgitation between 16 and 36 weeks, and to moderate regurgitation between 40 and 44 weeks, respectively. For these two animals, the stitch line for the sleeve was located at the middle of one leaflet, not along a commissure as in the other Gen2 valves, which was hypothesized as the reason for a gap forming at the same commissure. In PACV 20, the root diameter at the valve annulus had increased to 30.5 mm at 36 weeks. The explanted valve had all three leaflets intact. There was no gap between the leaflets at any of the commissures. Hyper-increase of the root diameter without commensurate leaflet growth presumably led to leaflets not closing during the cardiac cycle. In PACV21, there was a large gap at one commissure, which presumably was a cause for the insufficiency in addition to the hyper-increase of root diameter to 28 mm. However, tissue from the leaflets and roots of both valves all had comparable tensile properties to the implanted matrix.

The valved conduit of the Gen 3 valve was sutured directly to the pulmonary valve annulus. All valves exhibited only mild regurgitation after 52-week implantation, yielding improved Kaplan-Meier curves based on freedom from moderate regurgitation. All valves also exhibited a systolic pressure drop <10 mm Hg after 52-weeks. This valved conduit performance is consistent with the echo images at systole and diastole and Doppler echo, showing a conduit diameter that is uniform along the length of the conduit and matching the downstream native pulmonary artery.

Based on the echo images at 52 weeks, there was an increase in the length of the leaflet free edge. Gross imaging upon explantation showed that all three valved conduits possessed three intact leaflets each that appeared thin and pliable. No suture was evident, indicating the tubes had fused together prior to suture resorption. There was no change in thickness of the leaflets or their tensile mechanical properties during the 52-week implantation; their cellularity had increased substantially, but their collagen content was unchanged. The outflow portion of the conduit was thicker than at implantation, and, although the inflow portion thickness was unchanged, it was stiffer, likely correlating with an increased collagen concentration. The cellularity of both inflow and outflow portions of the conduit had increased substantially.

The histology for these valved conduits is consistent with the growth of the conduit concluded from echo measurements of diameter with substantial cellularity across the thickness and length of the conduit. The conduit tissue also appeared organized. The boundaries (initial surfaces) of the implanted tube were not clearly identifiable. Most notably, there was complete integration of the conduit with the connective tissue of the myocardial muscle fibers at the pulmonary annulus anastomosis, and there were no inflammatory cell foci, including a complete absence of mononuclear cells and no evidence of a foreign body response.

### Example 5—assessment of 4-tube (sleeved±Inflow Segment) Valved Conduit

A 3 cm hooded inflow segment created by using a longer sleeve was stitched to the Gen2 valve to create the Gen3 valved conduit, suitable for attachment to the pulmonary annulus as would be done for most RVOT repairs. The first animal implanted (PACV22) died the day of surgery due to apparent cardiac arrest resulting from the surgery, unrelated to the Gen3 valve. Two more animals (PACV23, 24) were successfully implanted with the Gen3 valved conduit and exhibited excellent valve function in the 1-week echo. They are followed by a third implantation for this cohort, with planned implantation and 52-week explant characterization.

### Example 6-19 mm Valved Conduit Manufacturing and Testing

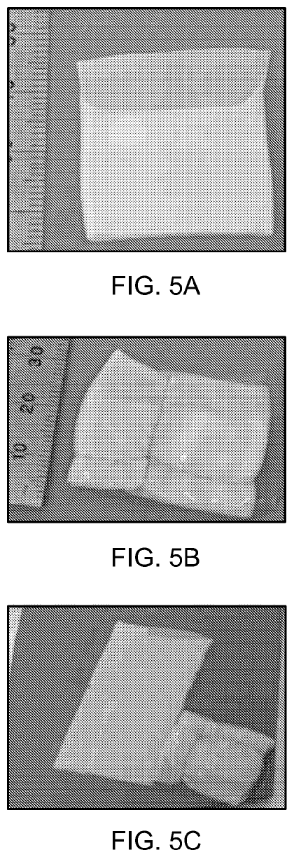

After 16 mm tubes are manufactured, the tubes are trimmed and stitched together into a tri-tube valve (Gen 1) as described in the first two process steps in FIG. 5. In the third step, the valve is placed within and at the center of a longer 22 mm tube (10 cm long) manufactured similarly to the 16 mm tubes, forming the valved conduit. The 22 mm diameter tube is appropriate for the tubes of matrix having thickness 600-800 um produced from ovine fibroblasts; the thinner tubes that result from human fibroblasts (˜400 μm) will make the appropriate inner diameter of the sleeve to be 21 mm. This “sleeve” possesses 5 mm of excess length on both ends, which is used to mount the valved conduit on a sterile fixture and evaluate for valve performance in custom aseptic cyclic tester. After each stitched line is formed, visual inspection is performed to ensure there are no gaps or suture holes compromised.

A valve testing apparatus for function evaluation is set up inside a laminar hood. The aseptic tester has two chambers, a sterile fluid chamber which is separated from the compressed air chamber with a silicone diaphragm. The cyclic air pressure is regulated using a sinusoidal valve and compressed in-house air at 20 psi. The valved conduit is mounted on the fixture is inserted in the test chamber. The chamber is then filled with phosphate buffer saline at room temperature. Cyclic pulsation at 70 bpm is started and amplitude is increased until the fluid pressure difference between the two transducers (one above and one below the valve) reads >10 mm Hg. End-on images are recorded at 240 fps. The images are analyzed for 1: asymmetry of leaflet geometry, 2: mismatch of leaflet motion (e.g. delayed closing of one leaflet), 3: prolapse, 4: pinwheel, and, 5: belly wrinkles. For each aspect, the properties are ranked as 0 for no observed defect, 1 for mild and 2 for moderate defect. Any valve with moderate defect on any property is rejected. Passed valves are removed from the fixture and 5 mm is trimmed from the sleeve at both ends (the portions used for mounting the conduit). The trimmed ends and the valved conduit are stored in the same product container in phosphate buffer saline for additional 7 days at 2-10° C. The trimmed ends are removed after 7 days, cut into segments and tested for sterility using a USP Immersion test along with mycoplasma and endotoxin testing. If negative tests are received, the valved conduit is released for clinical use.

### Example 7—Pediatric Valved Conduit From Biological Engineering in the Growing Lamb Model

A valved conduit with growth potential for pediatric patients remains an unmet need. Here we present a novel valved conduit with growth potential based on initial data in a growing lamb model. The valve is constructed from three biologically-engineered tubes of acellular collagenous matrix possessing somatic growth potential (Syedain et al., Nat Comms, 2016) using resorbable suture with each tube contributing to create a “leaflet” (Syedain et al, Sci Transl Med, 2021). This tri-tube valve previously demonstrated growth over 52 weeks when implanted interpositionally in the pulmonary artery.

In this study, a fourth tube of the same matrix is placed around the tri-tube valve to create a tubular valved conduit. The inlet segment is cut diagonally to allow for direct attachment to the myocardium, emulating a clinical repair.

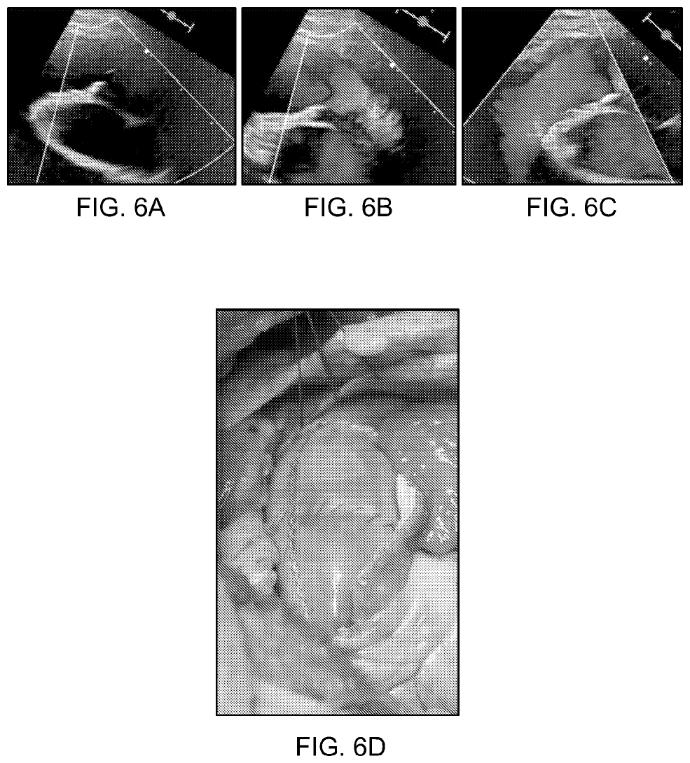

The first two lambs to date implanted in this study exhibited normal leaflet function, a pressure gradient less than 5 mm Hg without regurgitation or dilatation of the conduit at 12-week echocardiography (FIG. 6). During this time, the animal's weight increased from 34±2 kg to 48±1 kg. The inflow pattern with this direct myocardial implantation was more uniform than in the previous study that used a pulmonary artery interposition implant of the just the valve with no conduit.

Demonstrating integration of the conduit's matrix to the myocardium via recellularization is a key step toward a clinical trial. Previous pulmonary interposition implants of the valve, while achieving integration with the adjacent artery prior to dissolution of the Maxon suture (50% loss of tensile strength in ˜3 weeks, complete dissolution by ˜26 weeks), did not establish this clinically relevant point of myocardial integration. The preliminary finding is that the valved conduit exhibits excellent function at 12 weeks in the growing lamb and integration of the conduit's matrix with the valve annulus-myocardium will likely occur.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A pediatric valved conduit, comprising:
a biologically-engineered tubular conduit comprising a conduit inlet end, a conduit outlet end, and a conduit lumen therebetween;
a biologically-engineered valve comprising a valve inlet end and a valve outlet end, wherein the biologically-engineered valve is positioned in the conduit lumen,
wherein the valve inlet end of the biologically-engineered valve is fixedly attached in the lumen of the tubular conduit near the conduit inlet end and wherein the valve outlet end of the biologically-engineered valve is fixedly attached in the lumen of the tubular conduit near the conduit outlet end.

2. The pediatric valved conduit of claim 1, wherein the conduit inlet end and/or the conduit outlet end of the biologically-engineered tubular conduit extend(s) at least about 3 mm beyond the valve inlet end and/or the valve outlet end of the biologically-engineered valve, respectively.

3. The pediatric valved conduit of claim , wherein the biologically-engineered tubular conduit is about 8 mm to about 150 mm in length.

4. The pediatric valved conduit of claim 1, wherein the pediatric valved conduit is about 8 mm to about 150 mm in length.

5. The pediatric valved conduit of claim 1, wherein the diameter of the pediatric valved conduit is about 6 mm to about 24 mm.

6. The pediatric valved conduit of claim 1, claims, wherein one or both of the ends of the biologically engineered valve is/are fixedly attached in the lumen of the tubular conduit via sutures or stitches, staples, adhesives, double-sided adhesive tape, mechanical clips, crosslinking, chemical bonding, or thermal fusion

7. The pediatric valved conduit of claim 1, wherein one or both of the ends of the biologically engineered valve is/are fixedly attached circumferentially in the lumen of the tubular conduit

8. The pediatric valved conduit of claim 1, wherein the biologically engineered tubular conduit and/or the biologically engineered valve comprise(s) predominantly type I collagen and/or type III collagen.

9. The pediatric valved conduit of claim 8, wherein the type I collagen and/or type III collagen in the biologically engineered tubular conduit and/or the biologically engineered valve originates from thrombin, fibrinogen, and matrix-producing cells.

10. The pediatric valved conduit of claim 9, wherein the matrix-producing cells are dermal fibroblasts.

11. The pediatric valved conduit of claim 1, wherein the biologically engineered tubular conduit is a decellularized biologically engineered tubular conduit.

12. The pediatric valved conduit of claim 1, wherein the biologically engineered valve is a decellularized biologically engineered valve.

13. A method of repairing congenital heart defects, comprising: implanting the pediatric valved conduit into a pediatric subject suffering from a heart defect, wherein said pediatric valved conduit comprises:
a biologically-engineered tubular conduit comprising a conduit inlet end, a conduit outlet end, and a conduit lumen therebetween;
a biologically-engineered valve comprising a valve inlet end and a valve outlet end, wherein the biologically-engineered valve is positioned in the conduit lumen,
wherein the valve inlet end of the biologically-engineered valve is fixedly attached in the lumen of the tubular conduit near the conduit inlet end and wherein the valve outlet end of the biologically-engineered valve is fixedly attached in the lumen of the tubular conduit near the conduit outlet end.

14. The method of claim 13, wherein the congenital heart defect is Tetralogy of Fallot (TOF), pulmonary valve stenosis, bicuspid aortic valve, aortic stenosis, transposition of greater arteries, leaflet prolapse, any form of Right Ventricle Outflow Tract (RVOT) obstruction that requires reconstruction or any type of defective cardiac valve in need of repair.

15. The method of claim 13, wherein the implanted pediatric valved conduit increases in size as the pediatric subject increases in size.

16. The method of claim 13, wherein the pediatric subject is a neonate.

17. The method of claim 13, wherein the biologically engineered valve is selected from the group consisting of a mitral valve, an aortic valve, tricuspid valves, pulmonary heart valves, and vein valves.

18. The method of claim 13, wherein the valve is a bi-leaflet valve.

19. The method of claim 13, wherein the valve is a tri-leaflet valve.
